(19) United States
(12) Patent Application Publication
(10) Pub. No.: US 2022/0249745 A1
(43) Pub. Date: Aug. 11, 2022

(54) Engineered biodegradable vascular bioprostheses and production process thereof

(71) Applicant: Università degli Studi di Genova, Genova (IT)

(72) Inventors: Patrizia Perego, Genova (IT); Domenico Palombo, Genova (IT); Pier Francesco Ferrari, Genova (IT); Bahar Aliakbarian, Genova (IT); Bianca Pane, Genova (IT); Giovanni Salvatore Giuseppe Spinella, Genova (IT)

(21) Appl. No.: 17/627,960

(22) PCT Filed: Aug. 20, 2020
(86) PCT No.: PCT/IB2020/057829

§ 371 Date: Jan. 18, 2022

(30) Foreign Application Priority Data
Aug. 23, 2019 (IT) 102019000014985

(51) Int. Cl. A61L 31/04; A61L 31/16; A61L 31/10

(52) U.S. Cl. CPC A61L 31/041; A61L 2420/02; A61L 31/10; A61L 31/16

(57) ABSTRACT

Engineered biodegradable vascular bioprostheses include a polymeric construct containing a mixture of two polymers, poly (caprolactone) (PCL) and poly (glycerol sebacate) (PGS), which is functionalized with antioxidant bioactive molecules (biomolecules) that cause a modulation of the inflammation. The process for obtaining such engineered biodegradable vascular bioprostheses includes the preparation of a polymer solution by solubilizing the two polymers in mixtures of organic solvents, electrospinning the polymer solution, and bioengineering the prostheses.

The present invention relates to engineered biodegradable vascular bioprostheses and their production process.

The implantation of commercially available prostheses in patients suffering from peripheral obstructive vascular diseases generates in most cases an inflammatory-cicatricial process that involves the transformation of the prosthetic implant into a rigid and non-endothelialized structure, thus favoring thrombotic processes, especially for prostheses of small dimension and, in the case of the implantation of germs, in the course of bacteremia or septicemia, the impossibility of eradicating the infection without explant. To reduce and modulate the inflammatory and thrombotic responses, targeted therapeutic protocols are used. Furthermore, these commercial implants are not biodegradable, they are not bioactive and are not ideally suited for the replacement of small caliber vessels.

For these reasons, the use of biodegradable and engineered prostheses (bioprostheses) could ensure greater durability and patency of the implant over time. The success of the vascular bioprosthesis implantation is also strictly dependent on its biodegradability, i.e. the time interval during which it will perform its task before being replaced by a new vessel, without undergoing thrombus and calcification processes.

For these reasons, various methods for the construction of small caliber biodegradable vascular prostheses have been defined to date, still with little success. Among the manufacturing techniques, electrospinning with all its variants, 3D printing and decellurization of animal vessels are reported in the scarce literature about this subject.

Different biodegradable polymers have been extensively studied in the field of vascular tissue engineering with good results as regards their chemical-physical, mechanical and biological properties. The polymers used include natural polymers such as hyaluronic acid, gelatin and collagen and synthetic polymers such as poly (caprolactone) (PCL), poly (glycerol sebacate) (PGS), polylactic acid (PLLA) and poly-lactic acid-co-glycolide (PLGA). Recently, in the international scientific scenario, biomaterials are no longer considered only as a simple support in which cells must grow to form regenerated tissue, but also as drug delivery systems for bioactive molecules. This is made possible by the fact that the scaffolds can be functionalized with different compounds responsible for the prevention and modulation of inflammatory phenomena that are the basis of the failure of the implantation of bioprostheses, the recruitment of cells and the induction of their proliferation and/or of their trans-differentiation.

Currently, the technical problems regarding the implantation of prostheses in the field of vascular surgery are different:

impossibility of having small dimension prostheses to be used in the surgical treatment of the vessels of the upper and lower limbs;

impossibility of having small-dimension prostheses with greater patency over time (absence of early post-implantation thrombosis);

impossibility of having biodegradable prostheses that avoid subjecting the patient to repeated surgical interventions in case of treatment failure with the need to replace the prostheses currently on the market;

impossibility of having bioactive prostheses, or functionalized with anti-inflammatory biomolecules and trans-differentiation factors capable, respectively, of modulating the inflammatory process, a consequence of surgery, and inducing endothelialization of the construct used.

Engineered biodegradable vascular bioprostheses have now been obtained, synthesized by combining PCL and PGS, and functionalized with bioactive molecules, such as antioxidants with anti-inflammatory activity and proteins with cell trans-differentiation activity, with an internal diameter of less than 6 mm which, respectively, will be able to modulate the post-implantation inflammatory process and to induce an endothelialization of the implanted prosthesis.

PCL is one of the most widely used polymers for the synthesis of vascular prostheses, it is hydrophobic and biocompatible. PGS is an innovative, hydrophilic and also biocompatible polymer.

The combination of these polymers allows to add the advantages deriving from each of the two, obtaining bioabsorbable vascular prostheses that can be slowly degraded (reabsorbed) over time and replaced by a vascular neostructure.

The possibility of modulating the inflammatory process through the release of a bioactive molecule directly from a biodegradable prosthesis represents one of the main innovative and original features of this invention.

Another strong point of the described invention is the functionalization of bioprostheses with proteins capable of inducing the differentiation of monocytes into endothelial cells, favoring the endothelialization of the implanted prosthesis. The bioactive prosthesis, in the early stages of the implant, will be able to stem the inflammatory processes induced by the implant itself. This may be possible thanks to the release of the antioxidant and anti-inflammatory molecules with which the bioprostheses have been functionalized during the synthesis process. The endothelialization of the bioprosthesis, on the other hand, will be favored by the presence of proteins covalently linked to the internal surface of the construct which will direct the monocytes towards an endothelial cell line.

In addition, the originality of the invention presented also consists in the possibility of having small caliber bioprostheses available, preferably less than 6 mm in internal diameter, biodegradable, currently not available in the field of vascular surgery.

These functionalized bioprostheses can represent innovative medical devices that can respond to the inadequacy of synthetic prostheses currently used in vascular surgery.

Object of the present invention are engineered biodegradable vascular bioprostheses consisting of comprising a polymeric construct containing a mixture of two polymers, poly (caprolactone) (PCL) and poly (glycerol sebacate) (PGS), wherein bioactive molecules (biomolecules) are present choices among the antioxidants that exert an activity of modulation of inflammation.

The weight ratio between (caprolactone) (PCL) and poly (glycerol sebacate) (PGS) is preferably between 4 and 0.5, more preferably between 3 and 1.

As already reported, the resorption of the bioprosthesis can undergo strong interference due to the inflammatory response. This response can be modulated by various molecules such as antioxidants that exert an anti-inflammatory activity.

The antioxidants that exert an anti-inflammatory activity can preferably be selected from the polyphenols.

The latter represent a very wide class of compounds, chemically different from each other, which exert antioxidant and anti-inflammatory activity. A leading role is played by flavones, in particular apigenin, and by flavonoids, in particular quercetin, which are abundant in fruits and vegetables consumed daily. It has been shown that they are molecules with anti-inflammatory activity both in vitro and in vivo: in fact they are able to down-modulate the expression of cell adhesion molecules such as ICAM-1 (InterCellular Adhesion Molecule) and VCAM (Vascular Cell Adhesion) Molecule), induced by tumor necrosis factor α (TNF-α, Tumor Necrosis Factor-α), one of the pro-inflammatory cytokines most involved in the phlogisitic process. Furthermore, they appear to be bioactive towards the modulation of E-selectin (endothelial selectin), iNOS (inducible Nitric Oxide Synthase) and COX-2 (Cyclooxygenase-2), all molecules capable of mediating an inflammatory response.

Other classes of molecules that can be used having antioxidant and anti-inflammatory activities are phenols, in particular resveratrol and tyrosol.

The antioxidant in the polymer blend to be electro-spun must be in such a concentration that it is present, once released from the scaffold, in biologically active quantities.

Alongside the modulation of the inflammatory process, it is important that the implanted prosthesis is able to induce the endothelialization of its internal walls. This cellular process can be favored by the presence of molecules capable of inducing the differentiation of cells circulating in the blood into endothelial cells that will cover the scaffold wall.

For all the reasons mentioned above, the functionalization of biomaterials both with molecules with antioxidant and/or anti-inflammatory activity and with molecules with endothelial activity (for example capable of differentiating circulating monocytes in endothelial cells), represents a highly promising solution to problems that current prostheses present in the field of vascular surgery.

Therefore a linker can be conveniently present in biodegradable vascular bioprostheses that will allow the covalent bond between the functional groups of the polymer and the trans-differentiation protein factor, i.e. the linker that will be used must have a chemical group such as to be able to establish with the protein to be to bind a covalent bond.

These linkers will form a bridge between the polymer itself on which hydroxyl and/or carboxylic groups have been grafted and the protein that will be covalently linked to the linker and therefore to the polymer.

The linker used preferably contains hydroxyl and/or carboxylic groups, such as lysine.

The linker can also be not contained directly in the bioprosthesis, but grafted onto a possible coating of the bioprosthesis itself, such as gelatin, for the covalent bond with proteins.

A second object of the invention is the procedure for obtaining biodegradable vascular bioprostheses engineered in accordance with the invention.

In detail, the procedure for obtaining engineered vascular bioprostheses includes the following stages:

preparation of a polymeric solution by solubilizing the two polymers poly (caprolactone) (PCL) and poly (glycerol sebacate) (PGS) in mixtures of suitable organic solvents;

electrospinning of the obtained polymer solution;

bioengineering of vascular bioprostheses.

The preparation of the polymer solution is preferably carried out by separately preparing the solutions used for the polymer (PCL: PGS), the preparation of the polymeric solution of poly (caprolactone) (PCL) being carried out at a concentration between 10 and 30% (m/v) in a solution of suitable organic solvents, the preparation of the polymeric solution of poly (glycerol sebacate) (PGS) being carried out at a concentration between 10 and 30% (m/v) in a solution of suitable organic solvents, said two solutions obtained by being stirred, preferably by means of a magnetic stirrer, and then mixed.

The solution of suitable organic solvents for the preparation of the polymeric solution of poly (caprolactone) (PCL) and/or the solution of suitable organic solvents for the preparation of the polymeric solution of poly (glycerol sebacate) (PGS) preferably contains or consists of chloroform and ethanol in a ratio between 8/1 and 10/1, the ethanol used being able to contain dissolved bioactive molecules (biomolecules) chosen from among the antioxidants that exert an anti-inflammatory activity in one or both solutions.

The concentration in ethanol, in one or both solutions, of the bioactive molecules (biomolecules) chosen from among the antioxidants that exert an anti-inflammatory activity, if present, is preferably between 4 and 7 mg/mL, being able the concentration in the two solutions may also be different.

The bioactive molecules (biomolecules) are chosen among the antioxidants that exert an anti-inflammatory activity, preferably among the polyphenols, more preferably among the flavones, in particular apigenin, flavonoids, in particular quercetin, phenols, in particular resveratrol and tyrosol.

Quercetin is the preferred and recommended polyphenol.

Electrospinning shall be carried out in such a way as to obtain a polymeric construct that has suitable chemical-physical, mechanical, biological and suturability characteristics.

Said electrospinning stage takes place under the following preferred conditions:

flow of the electrospun solution between 0.60 and 2.20 mL/h;

voltage between 14 and 20 kVolt;

external diameter of the collector between 1 and 6 mm;

collector needle distance between 15 and 18 cm;

volume of the electrospun solution between 1.5 and 2.0 mL;

collector rotation speed between 400 and 800 rpm;

collector translation speed between 350 and 650 mm/min.

By varying the parameters of the electrospinning process within the ranges indicated above, it is possible to obtain bioprostheses, varying between 1 and 6 mm in diameter, with the best possible performances in terms of chemical-physical, mechanical, suturability and biological activity properties.

The bioengineering of the obtained bioprostheses can also take place using a linker allowing the covalent bond between the functional groups of the polymer and the trans-differentiation protein factor.

The bioengineering can be either simultaneous to the electrospinning stage, see for example the addition of the antioxidant to the two polymers or the addition in the polymeric solution of nanoparticles that encapsulate or polyphenols or trans-differentiation proteins, or subsequent to the electrospinning stage.

The bioengineering of the obtained bioprostheses, carried out downstream of the electrospinning step of the polymeric solution, preferably takes place by coating the bioprosthesis obtained with a compound, such as gelatin, and by grafting the aforementioned linker containing hydroxyl and/or carboxylic groups so as to coat the inner surface of the bioprosthesis.

A further object of the invention is represented by the use of vascular bioprostheses engineered as described above as medical devices.

The invention claimed is:

1. Engineered biodegradable vascular bioprostheses comprising:
a polymeric construct containing a mixture of two polymers consisting of poly (caprolactone) (PCL) and poly (glycerol sebacate) (PGS); and
bioactive molecules (biomolecules) selected among the antioxidants which exert an anti-inflammatory activity.

2. The engineered biodegradable vascular bioprostheses according to claim 1, wherein a weight ratio between the poly (caprolactone) (PCL) and poly (glycerol sebacate) (PGS) is between 4 and 0.5.

3. The engineered biodegradable vascular bioprostheses according to claim 1, wherein the antioxidants are polyphenols.

4. The engineered biodegradable vascular bioprostheses according to claim 3, wherein the polyphenols are selected among the group consisting of flavones, apigenin, flavonoids, and phenols.

5. The engineered biodegradable vascular bioprostheses according to claim 1, wherein the polymeric construct comprises a linker allowing a covalent bonding between functional groups of at least one of the two polymers and a trans-differentiation protein factor.

6. The engineered biodegradable vascular bioprostheses according to claim 5, wherein the linker contains hydroxyl and/or carboxyl groups.

7. The engineered biodegradable vascular bioprostheses according to claim 6, further comprising gelatin providing a coating of said bioprostheses, in which the linker containing hydroxyl and/or carboxyl groups is grafted.

8. A process for obtaining engineered biodegradable vascular bioprostheses according to claim 1, comprising:
preparing a polymer solution by solubilization of the two polymers, the two polymers being the poly (caprolactone) (PCL) and the poly (glycerol sebacate) (PGS), in a mixture of an organic solvent;
electrospinning the polymer solution;
bioengineering the polymer solution to obtain the engineered biodegradable vascular bioprostheses.

9. The process according to claim 8, wherein preparing the polymer solution comprises separately preparing a solutions for a polymer (PCL:PGS), wherein a first polymer solution of poly (caprolactone) (PCL) is carried out at a concentration between 10 and 30% (w/v) in a solution of an organic solvent, wherein a second a polymer solution of poly (glycerol sebacate) (PGS) is carried out at a concentration between 10 and 30% (w/v) in a solution of an organic solvent, said first and said second polymer solutions being stirred and then mixed.

10. The process according to claim 9, wherein one or both of the first and the second polymer solution contains or is constituted by chloroform and ethanol in a ratio between 8/1 and 10/1, the ethanol, used in one or both polymer solutions, containing dissolved-inside bioactive molecules (biomolecules) selected among the antioxidants which exert an anti-inflammatory activity.

11. The process according to claim 10, wherein a concentration in ethanol, in one or both of the first and the second polymer solutions, of the bioactive molecules (biomolecules) selected among the antioxidants which exert an anti-inflammatory activity is between 4 and 7 mg/ml, said concentration being different in the first and the second polymer solutions if so desired.

12. The process according to claim 10, wherein the bioactive molecules (biomolecules) selected among the antioxidants which exert an inflammation activity are polyphenols flavonoids, or phenol.

13. The process according to claim 12, wherein the polyphenol is quercetin.

14. The process according to claim 8, wherein the electrospinning occurs under the following conditions:
flow of electrospun solution between 0.60 and 2.20 ml/h;
voltage between 14 and 20 kVolt;
outside diameter of a collector between 1 and 6 mm;
collector-needle distance between 15 and 18 cm;
volume of an electrospun solution between 1.5 and 2.0 ml;
collector rotation speed between 400 and 800 rpm; and
collector translation speed between 350 and 650m/min.

15. The process according to claim 8, wherein the bioengineering comprises adding a linker which provides for a covalent bonding between functional groups of the polymer in the polymer solution and a trans-differentiation protein factor.

16. The process according to claim 8, wherein the bioengineering is performed at a same time as the electrospinning.

17. The process according to claim 8, wherein the bioengineering is carried out downstream of the electrospinning of the polymer solution.

18. The process according to claims 15, wherein the bioengineering is carried out downstream of the electrospinning, and wherein the bioengineering occurs by coating a bioprosthesis with a compound, and by grafting a linker containing hydroxyl and/or carboxyl groups so to cover an inner surface of the bioprosthesis.

19. Engineered biodegradable vascular bioprostheses according to claim 1, wherein the engineered biodegradable vascular bioprostheses are configured as medical devices for obstructive vascular diseases.
